(19) United States
(12) Patent Application Publication
(10) Pub. No.: US 2024/0269121 A1
(43) Pub. Date: Aug. 15, 2024

(54) METHODS OF REDUCING ACNE AND ROSACEA RELAPSE RATE AND SEVERITY

(71) Applicant: Sol-Gel Technologies Ltd., Ness Ziona (IL)

(72) Inventors: Moshe ARKIN, Kfar Shmaryahu (IL); Ofer TOLEDANO, Kfar Saba (IL); Karine NEIMANN, Ness Ziona (IL); Hila HAKAK DJERBI, Tel-Aviv (IL)

(73) Assignee: Sol-Gel Technologies Ltd., Ness Ziona (IL)

(21) Appl. No.: 18/567,464

(22) PCT Filed: Jun. 8, 2022
(86) PCT No.: PCT/IL2022/050613

§ 371 Date: Dec. 6, 2023

Related U.S. Application Data
(60) Provisional application No. 63/208,108, filed on Jun. 8, 2021.

(51) Int. Cl. A61K 31/44; A61K 31/05; A61K 31/194; A61K 31/196; A61K 31/202; A61K 31/277; A61K 31/327; A61K 31/4035; A61K 31/404; A61K 31/4164; A61K 31/42; A61K 31/421; A61K 31/427; A61K 31/437; A61K 31/4545; A61K 31/473; A61K 31/519; A61K 31/65; A61K 31/69; A61K 31/7048; A61K 45/06

(52) U.S. Cl. CPC A61K 31/44; A61K 31/05; A61K 31/194; A61K 31/196; A61K 31/202; A61K 31/277; A61K 31/327; A61K 31/4035; A61K 31/404; A61K 31/4164; A61K 31/42; A61K 31/421; A61K 31/427; A61K 31/437; A61K 31/4545; A61K 31/473; A61K 31/519; A61K 31/65; A61K 31/69; A61K 31/7048; A61K 45/06

(57) ABSTRACT

The present invention relates to a method of reducing acne relapse or rosacea relapse rate or severity, by reducing the skin's immunological response, wherein the method comprises administering a topical composition comprising at least one of a PDE4 inhibitor, a JAK inhibitor, an AhR modulator, or any combination thereof.

### FIELD OF THE INVENTION

The present invention relates to a method of reducing acne relapse or rosacea relapse rate or severity, by reducing the skin's immunological response, wherein the method comprises administering a topical composition comprising at least one of a PDE4 inhibitor, a JAK inhibitor, an AhR modulator, or any combination thereof.

### BACKGROUND OF THE INVENTION

Acne is a chronic inflammatory disease of the pilosebaceous unit resulting from androgen-induced increased sebum production, altered keratinization, inflammation, and bacterial colonisation of hair follicles on the face, neck, chest, and back by Propionibacterium acnes. Although early colonisation with Propionibacterium acnes and family history might have important roles in the disease, exactly what triggers acne and how treatment affects the course of the disease remain unclear (Williams H. C. et al., The Lancet, Vol. 379. January 2012, pp. 361-372).

Acne is caused by four major factors: (1) production of oil by enlarged oil glands in the skin, (2) blockage of the hair follicles that release oil, (3) growth of bacteria, called Propionibacterium acnes (P. acnes), within the hair follicles, and (4) inflammatory/immune response to P. acnes.

There is no ideal treatment for acne, although a suitable regimen for reducing lesions can be found for most patients. Good quality evidence on comparative effectiveness of common topical and systemic acne therapies is scarce. Topical therapies including benzoyl peroxide, retinoids, and antibiotics when used in combination usually improve control of mild to moderate acne. Treatment with combined oral contraceptives can help women with acne. Patients with more severe inflammatory acne usually need oral antibiotics combined with topical benzoyl peroxide to decrease antibiotic-resistant organisms.

Oral isotretinoin is the most effective acne therapy and is used early in severe disease, although its use is limited by teratogenicity and other side-effects.

Acne vulgaris (cystic acne or “acne”) is a common human skin disease, characterized by areas of skin with redness, comedones (blackheads and whiteheads), papules (pinheads), pustules (pimples), nodules (large papules) and possibly scarring. Acne affects mostly skin with the densest population of sebaceous follicles; these areas include the face, the upper part of the chest, and the back. Severe acne is inflammatory, but acne can also manifest in noninflammatory forms. Acne occurs most commonly during adolescence, and often continues into adulthood.

Rosacea is a chronic disease of inflammatory dermatitis that mainly affects the median part of the face and the eyelids of certain adults. It is characterized by telangiectatic erythema, dryness of the skin, papules and pustules. Conventionally, rosacea develops in adults from the ages of 30 to 50; it more frequently affects women, although the condition is generally more severe in men. Rosacea is a primitively vascular condition whose inflammatory stage lacks the cysts and comedones characteristic of common acne.

Factors that have been described as possibly contributing towards the development of rosacea include for example: the presence of parasites such as the Demodex folliculorum, the presence of bacteria such as Helicobacter pylori (a bacterium associated with gastrointestinal disorders), hormonal factors (such as endocrine factors), climatic and immunological factors, and so forth.

Rosacea develops in four stages over several years, in spasms aggravated by variations in temperature, alcohol, spices, exposure to sunlight and stress.

The various stages of the disease are the following:

Stage 1: stage of erythema episodes. The patients have erythrosis spasms due to the sudden dilation of the arterioles of the face, which then take on a congestive, red appearance. These spasms are caused by the emotions, meals and temperature changes.

Stage 2: stage of couperosis, i.e., of permanent erythema with telangiectasia. Certain patients also have oedema on the cheeks and the forehead.

Stage 3: inflammatory stage (papular-pustular rosacea) with appearance of inflammatory papules and pustules, but without affecting the sebaceous follicles and thus with absence of cysts and comedones.

Stage 4: rhinophyma stage. This late phase essentially affects men. The patients have a bumpy, voluminous red nose with sebaceous hyperplasia and fibrous reordering of the connective tissue.

Typical treatments of rosacea include oral or topical administration of antibiotics such as tetracyclines, salicylic acid, anti-fungal agents, steroids, metronidazole, isotretinoin in severe cases, or anti-infectious agents such as azelaic acid.

US 20110052515 describes a topically applicable formulation for treating rosacea, comprising at least one avermectin compound and benzoyl peroxide (BPO, an anti-acne agent).

Montes et al. (Cutis, 32, 185-190 (1983)) disclosed the use of BPO dissolved in acetone gel formulation for the treatment of rosacea.

Phosphodiesterase-4 inhibitor (also known as “PDE4 inhibitor”) is a drug used to block the degradative action of phosphodiesterase 4 (PDE4) on cyclic adenosine monophosphate (cAMP) having anti-asthmatic and anti-inflammatory activity.

Phosphodiesterase-4 (PDE4) inhibitors have the potential to modulate immune responses from the Th1 toward the Th2 phenotype and are considered candidate therapies for Th1-mediated autoimmune disorders. (Bielekova B. et al. Therapeutic potential of phosphodiesterase-4 and -3 inhibitors in Th1-mediated autoimmune diseases. J Immunol. 2000 Jan. 15; 164(2):1117-24). Non-limiting examples of PDE4 inhibitors include roflumilast, apremilast, crisaborole, rolipram, cilomilast, ibudilast, piclamilast and combinations thereof.

Janus kinase inhibitors, also known as JAK inhibitors or jakinibs (henceforth JAK inhibitors or JAKi), are a class of drugs interfering with the JAK-STAT signaling pathway by inhibiting at least one of the Janus kinase enzymes JAK1, JAK2, JAK3 or TYK2. Some JAK inhibitors inhibit all the above enzymes and are therefore named pan-JAK inhibitors.

JAK-STAT is an intracellular signaling pathway upon which many different pro-inflammatory signaling pathways converge (Damsky W. J Am Acad Dermatol. 2017 April; 76(4): 736-744).

A small number of JAK inhibitors are already marketed in the US as oral drugs for the treatment of conditions like rheumatoid arthritis, psoriatic arthritis, ulcerative colitis (Xelianz), myelofibrosis and polycythemia vera (Jakafi).

No topical JAK inhibitor is presently marketed. The Janus family kinases (Jaks), JAK1, JAK2, JAK3, and Tyk2, form one subgroup of the non-receptor protein tyrosine kinases. They are involved in cell growth, survival, development, and differentiation of a variety of cells but are critically important for immune cells and hematopoietic cells (Ghoreschi K, et al. Janus kinases in immune cell signaling. Immunol Rev. 2009; 228(1):273-287). Non-limiting examples of JAK inhibitors tofacitinib, abrocitinib, ruxolitinib, delgocitinib, oclacitinib, baricitinib, peficitinib, Ati 1777 and combinations thereof.

The aryl hydrocarbon receptor (AhR) is a ligand-activated transcription factor that is activated by small molecules provided by the diet, microorganisms, metabolism and pollutants. The AhR was found to play an intrinsic role in survival and proliferation of immune cells and is essential for murine skin barrier integrity of the epidermis (J. Invest Dermatol. 2016 November; 136(11):2260-2269). The AhR provides a molecular pathway through which environmental factors modulate the immune response in health and disease. (Quintana F J, Sherr D H. Aryl hydrocarbon receptor control of adaptive immunity. Pharmacol Rev. 2013; 65(4):1148-1161).

Non-limiting examples of AhR include tapinarof, FICZ (6-formylindolo(3,2b)carbazole), Indirubin, ITE (2-(1′H-indole-3′carbonyl)-thiazole-4-carboxylic acid methyl ester), L-Kynurenine, a Flavonoid, Leflunomide, Norisoboldine, and combinations thereof.

### SUMMARY OF THE INVENTION

The present invention provides a method of reducing acne relapse or rosacea relapse rate, or reducing the severity of acne relapse or rosacea relapse in a subject, wherein the method comprises administering a topical composition comprising at least one of a PDE4 inhibitor, a JAK inhibitor, an AhR modulator, or any combination thereof; wherein the method reduces the skin's immunological response and thereby, reduces acne or rosacea relapse rate, or reduces the severing of acne or rosacea relapse.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, the present invention provides a method of reducing acne relapse or rosacea relapse rate, or reducing the severity of acne relapse or rosacea relapse in a subject, wherein the method comprises administering a topical composition comprising at least one of a PDE4 inhibitor, a JAK inhibitor, an AhR modulator, or any combination thereof. In some embodiments, the method reduces the skin's immunological response and thereby, reduces acne or rosacea relapse rate, or reduces the severing of acne or rosacea relapse.

Without being bound to any theory, acne vulgaris and rosacea are chronic immune mediated inflammation of the pilosebaceous unit. There are many therapeutic options that address the various elements in the acne cascade: androgen production, sebaceous gland hyperactivity, P. acnes, keratinocytes hyperproliferation; or of rosacea. Unfortunately, none of these modalities of treatments, including oral antibiotics and oral isotretinoin, can prevent relapse of for example acne. Relapse rate have been reported from 10% to 50%. Relapses have obviously not only a medical problem but they are psychologically very stressful to patients, particularly adolescents. Thus, the need for safe and efficacious treatment of acne relapsed and rosacea relapsed is very high.

Theoretically, any present treatment can be used as maintenance therapy, but in practice none of them is optimal: very long exposure to antibiotics may create bacterial resistance. Benzoyl peroxide and azelaic acid are irritating and local retinoids, which are quite effective for acne as maintenance therapy, require avoidance of exposure to the sun, which is not feasible for prolonged periods. This invention proposes the use of topical treatments with agents which have not necessarily shown impressive efficacy when acne/rosacea is fully development but are instrumental in reducing skin immune response to the acne/rosacea triggering process and consequently avoid or delay or reduce the severity of the relapse.

In some embodiments, the present invention provides a method of reducing acne relapse rate in a subject, wherein the method comprises administering a topical composition comprising at least one of a PDE4 inhibitor, a JAK inhibitor, an AhR modulator, or any combination thereof, wherein the method reduces the skin's immunological response and thereby, reduces acne relapse rate.

In some embodiments, the present invention provides a method of reducing the severity of acne relapse, wherein the method comprises administering a topical composition comprising at least one of a PDE4 inhibitor, a JAK inhibitor, an AhR modulator, or any combination thereof, wherein the method reduces the skin's immunological response and thereby, reduces the severity of acne relapse.

In some embodiments, the present invention provides a method of reducing rosacea relapse rate, wherein the method comprises administering a topical composition comprising at least one of a PDE4 inhibitor, a JAK inhibitor, an AhR modulator, or any combination thereof, wherein the method reduces the skin's immunological response and thereby, reduces rosacea relapse rate.

In some embodiments, the present invention provides a method of reducing the severity of rosacea relapse in a subject, wherein the method comprises administering a topical composition comprising at least one of a PDE4 inhibitor, a JAK inhibitor, an AhR modulator, or any combination thereof, wherein the method reduces the skin's immunological response and thereby, reduces the severing of rosacea relapse.

In some embodiments, the present invention provides a method of reducing acne relapse or rosacea relapse rate, or reducing the severity of acne relapse or rosacea relapse in a subject, wherein the method comprises administering a topical composition comprising at least one of a PDE4 inhibitor, a JAK inhibitor, an AhR modulator, or any combination thereof, wherein the topical composition comprises at least one PDE4 inhibitor at a concentration of between 0.05 wt %-0.1 wt %. In other embodiments, the topical composition comprises at least one PDE4 inhibitor at a concentration of 0.05 wt %, 0.06 wt %, 0.07 wt %, 0.08 wt %, 0.09 wt % or 0.1 wt %. In other embodiments, the method reduces the skin's immunological response and thereby, reduces acne or rosacea relapse rate, or reduces the severing of acne or rosacea relapse.

In some embodiments, the present invention provides a method of reducing acne relapse or rosacea relapse rate, or reducing the severity of acne relapse or rosacea relapse in a subject, wherein the method comprises administering a topical composition comprising at least one of a PDE4 inhibitor, a JAK inhibitor, an AhR modulator, or any combination thereof, wherein the topical composition comprises at least one AhR modulator at a concentration of between 0.01-0.5 wt %. In other embodiments, the topical composition comprises at least one AhR modulator at a concentration of between 0.01 wt %-0.05 wt %, or between 0.01 wt %-0.1 wt %, or between 0.01 wt % to 0.2 wt %, or between 0.1 wt % to 0.5 wt % or between 0.1 wt % to 0.2 wt %, or between 0.1 wt % to 0.3 wt %, or between 0.2 wt % to 0.5 wt %. In other embodiments, the method reduces the skin's immunological response and thereby, reduces acne or rosacea relapse rate, or reduces the severing of acne or rosacea relapse.

In some embodiments, the present invention provides a method of reducing acne relapse or rosacea relapse rate, or reducing the severity of acne relapse or rosacea relapse in a subject, wherein the method comprises administering a topical composition comprising at least one of a PDE4 inhibitor, a JAK inhibitor, an AhR modulator, or any combination thereof, wherein the topical composition comprises at least one JAK inhibitor at a concentration less than 3 wt %. In other embodiment, the topical composition comprises at least one JAK inhibitor at a concentration of less than 2 wt %. In other embodiment, the topical composition comprises at least one JAK inhibitor at a concentration of between 0.01 wt % to 3 wt %, or between 0.01 wt % to 0.05 wt %, or between 0.01 wt % to 0.1 wt %, or between 0.1 wt % to 1 wt %, or between 0.1 wt % to 2 wt % or between 0.1 wt % to 3 wt %, or between 1 wt % to 3 wt %. In other embodiments, the method reduces the skin's immunological response and thereby, reduces acne or rosacea relapse rate, or reduces the severing of acne or rosacea relapse.

In some embodiments, the present invention provides a method of reducing acne relapse or rosacea relapse rate, or reducing the severity of acne relapse or rosacea relapse in a subject, wherein the method comprises administering a topical composition comprising at least one PDE4 inhibitor at a concentration of between 0.05 wt %-0.1 wt %, wherein the method reduces the skin's immunological response and thereby, reduces acne or rosacea relapse rate, or reduces the severing of acne or rosacea relapse. In other embodiments, the topical composition comprises at least one PDE4 inhibitor at a concentration of 0.05 wt %, 0.06 wt %, 0.07 wt %, 0.08 wt %, 0.09 wt % or 0.1 wt %.

In some embodiments, the present invention provides a method of reducing acne relapse or rosacea relapse rate, or reducing the severity of acne relapse or rosacea relapse in a subject, wherein the method comprises administering a topical composition comprising at least one AhR modulator at a concentration of between 0.01-0.5 wt %, wherein the method reduces the skin's immunological response and thereby, reduces acne or rosacea relapse rate, or reduces the severing of acne or rosacea relapse. In other embodiments, the topical composition comprises at least one AhR modulator at a concentration of between 0.01 wt %-0.05 wt %, or between 0.01 wt %-0.1 wt %, or between 0.01 wt % to 0.2 wt %, or between 0.1 wt % to 0.5 wt % or between 0.1 wt % to 0.2 wt %, or between 0.1 wt % to 0.3 wt %, or between 0.2 wt % to 0.5 wt %.

In some embodiments, the present invention provides a method of reducing acne relapse or rosacea relapse rate, or reducing the severity of acne relapse or rosacea relapse in a subject, wherein the method comprises administering a topical composition comprising at least one JAK inhibitor a concentration of less than 3 wt %, wherein the method reduces the skin's immunological response and thereby, reduces acne or rosacea relapse rate, or reduces the severing of acne or rosacea relapse. In other embodiment, the topical composition comprises at least one JAK inhibitor at a concentration of between 0.01 wt % to 3 wt %, or between 0.01 wt % to 0.05 wt %, or between 0.01 wt % to 0.1 wt %, or between 0.1 wt % to 1 wt %, or between 0.1 wt % to 2 wt % or between 0.1 wt % to 3 wt %, or between 1 wt % to 3 wt %.

In some embodiments, the present invention provides a method of reducing acne relapse or rosacea relapse rate, or reducing the severity of acne relapse or rosacea relapse in a subject, wherein the method comprises administering a topical composition comprising at least one PDE4 inhibitor at a concentration of between 0.05 wt %-0.1 wt % and at least one AhR modulator at a concentration of between 0.01-0.5 wt % and at least one JAK inhibitor a concentration of less than 3 wt %. In other embodiments, the method reduces the skin's immunological response and thereby, reduces acne or rosacea relapse rate, or reduces the severing of acne or rosacea relapse.

In some embodiments, the present invention provides a method of reducing acne relapse or rosacea relapse rate, or reducing the severity of acne relapse or rosacea relapse in a subject, wherein the method comprises administering a topical composition comprising at least one PDE4 inhibitor at a concentration of between 0.05 wt %-0.1 wt % and at least one AhR modulator at a concentration of between 0.01-0.5 wt %. In other embodiments, the method reduces the skin's immunological response and thereby, reduces acne or rosacea relapse rate, or reduces the severing of acne or rosacea relapse.

In some embodiments, the present invention provides a method of reducing acne relapse or rosacea relapse rate, or reducing the severity of acne relapse or rosacea relapse in a subject, wherein the method comprises administering a topical composition comprising at least one PDE4 inhibitor at a concentration of between 0.05 wt %-0.1 wt % and at least one JAK inhibitor a concentration of less than 3 wt %. In other embodiments, the method reduces the skin's immunological response and thereby, reduces acne or rosacea relapse rate, or reduces the severing of acne or rosacea relapse.

In some embodiments, the present invention provides a method of reducing acne relapse or rosacea relapse rate, or reducing the severity of acne relapse or rosacea relapse in a subject, wherein the method comprises administering a topical composition comprising at least one AhR modulator at a concentration of between 0.01-0.5 wt % and at least one JAK inhibitor a concentration of less than 3 wt %. In other embodiments, the method reduces the skin's immunological response and thereby, reduces acne or rosacea relapse rate, or reduces the severing of acne or rosacea relapse.

In other embodiments, the at least one PDE4 inhibitor used in the methods of this invention is selected from roflumilast, apremilast, piclamilast, ibudilast, crisaborole, difamilast, rolipram, cilomilast, their salts, hydrates or solvates, and combinations thereof. Each represents a separate embodiment of this invention. In some embodiments, the at least one PDE4 inhibitor is roflumilast.

In some embodiments, the at least one JAK inhibitor used in the methods of this invention is selected from tofacitinib, abrocitinib, ruxolitinib, deuterated ruxolitinib, upadacitinib, delgocitinib, oclacitinib, baricitinib, peficitinib, Ati 1777 or salts thereof, and combinations thereof; Each represents a separate embodiment of this invention. In some embodiments, the at least one JAK inhibitor is ruxolitinib.

In some embodiments, the at least one AhR modulator, used in the methods of this invention is selected from tapinarof, FICZ (6-formylindolo(3,2b)carbazole), Indirubin, ITE (2-(1′H-indole-3′carbonyl)-thiazole-4-carboxylic acid methyl ester), L-Kynurenine, a Flavonoid, Leflunomide, Norisoboldine, and any combination thereof. Each represents a separate embodiment of this invention. In some embodiments, the at least one AhR modulator is tapinarof.

In some embodiments, the topical composition administered according to the method of the present invention, is in the form of a cream, an ointment, a spray, a gel, a lotion, a spray, a shampoo, a patch, or a foam, each represents a separate embodiment of this invention.

In some embodiments, the method of the present invention reduces the reoccurrence of acne or rosacea by 30%-300%. In some embodiments, the method of the present invention reduces the reoccurrence of acne or rosacea by 30%-100%. In some embodiments, the method of the present invention reduces the reoccurrence of acne or rosacea by 30%-200%. In other embodiments, the methods described herein reduces the reoccurrence of acne or rosacea by 30%-50%. In other embodiments, the methods described herein reduces the reoccurrence of acne or rosacea by 50%-70%. In other embodiments, the methods described herein reduces the reoccurrence of acne or rosacea by 50%-100%. In other embodiments, the methods described herein reduces the reoccurrence of acne or rosacea by 30%, 40%, 50%, 60%, 70%, 80%, 90%, 100%, 200% or 300%. The recurrence of acne or rosacea is defined by the period the acne or rosacea is relapse/reoccurs after remission (i.e. achieved IGA success). For example, reduction of 30% reoccurrence refers to additional 30% time between the remission (i.e. achieved IGA success) and the reoccurrence of the disease.

In some embodiments, the method of the present invention prevents the reoccurrence of acne or rosacea and their symptoms.

Recurrence of acne or rosacea refers to the occurrence of acne or rosacea or symptoms thereof after remission (i.e. achieved IGA success). The reduction in reoccurrence or relapse of acne or rosacea according to this invention is substantially reduced for example from time of relapse of about two weeks, three weeks, one months, two months, three months with no treatment to relapse period of about four weeks, six weeks, two months four months, six months, respectively with the treatment of this invention. In other embodiments, the present invention provides a relapse period of about four months, five months, six months, seven months, eight months, nine months, 10 months or more following the treatment of this invention, compared to a relapse period of about three months with no treatment of this invention.

In some embodiments, the method reduces the severity of acne relapse or rosacea relapsed by 30%-100% as measured by the number of inflammatory lesion count for rosacea and by the number of inflammatory lesion count for acne. In some embodiments, the method reduces the severity of acne relapse or rosacea relapsed by 30%, 40%, 50%, 60%, 70%, 80%, 90% or 100% as measured by the number of inflammatory lesion count for rosacea and by the number of inflammatory lesion count for acne.

### Regimen of Administration

According to some embodiments, the topical composition comprising at least one of a PDE4 inhibitor, a JAK inhibitor, an AhR modulator, or combinations thereof, is administered for a period of between one month to 24 months during, before or after acne treatment or rosacea treatment. In some embodiments, the acne treatment comprises treating the subject by administering an anti-acne agent.

In some embodiments, the anti-acne agent comprises benzoyl peroxide, azelaic acid, Roaccutane, retinoids, antibiotics or combinations thereof.

In some embodiments, the topical composition comprising at least one of a PDE4 inhibitor, a JAK inhibitor, an AhR modulator or combinations thereof is administered for a period of between one month to 24 months during, before or after rosacea treatment. In some embodiments, the rosacea treatment comprises treating the subject by administering an anti-rosacea agent.

In some embodiments, the rosacea agent comprises benzoyl peroxide, metronidazole, erythromycin, azelaic acid, ivermectin, doxycycline, minocycline, or combinations thereof.

### Definitions

Unless defined otherwise, all technical and scientific terms used herein have the same meaning as is commonly understood by one of skill in the art to which the invention pertains. In case of conflict, the specification, including definitions, takes precedence.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “modulator” and “agonist” are used herein interchangeably.

Whenever a numerical range is indicated herein, it is meant to include any cited numeral (fractional or integral) within the indicated range. The phrases “between” a first indicate number and a second indicate number and “from” a first indicate number “to” a second indicate number are used herein interchangeably and are meant to include the first and second indicated numbers and all the fractional and integral numerals therebetween.

The terms “comprise”, “comprising”, “includes”, “including”, “having” and their conjugates mean “including but not limited to”.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

1. A method of reducing acne relapse or rosacea relapse rate, or reducing the severity of acne relapse or rosacea relapse in a subject, wherein the method comprises administering a topical composition comprising at least one of a PDE4 inhibitor, a JAK inhibitor, an AhR modulator, or any combination thereof; wherein the method reduces the skin's immunological response and thereby, reduces acne or rosacea relapse rate, or reduces the severing of acne or rosacea relapse.

2. The method of claim 1, wherein the at least one PDE4 inhibitor is selected from roflumilast, apremilast, piclamilast, ibudilast, crisaborole, difamilast, cilomilast, their salts, hydrates or solvates, and combinations thereof.

3. The method of claim 2, wherein the PDE4 inhibitor is roflumilast.

4. The method of claim 1, wherein the topical composition comprises at least one PDE4 inhibitor at a concentration of between 0.05 wt % and 0.1 wt %.

5. The method of claim 1, wherein the at least one JAK inhibitor is selected from tofacitinib, abrocitinib, ruxolitinib, deuterated ruxolitinib, upadacitinib, delgocitinib, oclacitinib, baricitinib, peficitinib, Ati 1777 or salts thereof, and combinations thereof.

6. The method of claim 5, wherein the JAK inhibitor is in a concentration of less than 3 wt %.

7. The method of claim 1, wherein the at least one AhR modulator is selected from tapinarof, FICZ (6-formylindolo(3,2b)carbazole), Indirubin, ITE (2-(1′H-indole-3′carbonyl)-thiazole-4-carboxylic acid methyl ester), L-Kynurenine, a Flavonoid, Leflunomide, Norisoboldine, and any combination thereof.

8. The method of claim 7, wherein the at least one AhR modulator is tapinarof.

9. The method of claim 1, wherein the topical composition comprises at least one AhR modulator at a concentration of between 0.01-0.5 wt %.

10. The method of claim 1, wherein the topical composition comprising at least one of a PDE4 inhibitor, a JAK inhibitor, an AhR modulator, or combinations thereof is administered for a period of between one month to 24 months during, before or after acne treatment.

11. The method of claim 10, wherein the acne treatment comprises treating the subject by administering an anti-acne agent.

12. The method of claim 10, wherein the anti-acne agent, comprises benzoyl peroxide, azelaic acid, Roaccutane, retinoids, antibiotics or combinations thereof.

13. The method of claim 1, wherein the topical composition comprising at least one of a PDE4 inhibitor, a JAK inhibitor, an AhR modulator or combinations thereof is administered for a period of between one month to 24 months during, before or after rosacea treatment.

14. The method of claim 13, wherein the rosacea treatment comprises treating the subject by administering an anti-rosacea agent.

15. The method of claim 14, wherein the rosacea agent comprises benzoyl peroxide, metronidazole, erythromycin, azelaic acid, ivermectin, doxycycline, minocycline, or combinations thereof.

16. The method of claim 1, wherein the topical composition is in the form of a cream, an ointment, a spray, a gel, a lotion, a spray, a shampoo, a patch, or a foam.

17. The method of claim 1, wherein the reoccurrence of acne or rosacea is reduced by 30%-100%.

18. The method of claim 1, wherein the severity of acne relapse or rosacea relapse is reduced by 30%-100% as measured by the number of inflammatory lesion count.
